(19) United States
(12) Patent Application Publication
(10) Pub. No.: US 2009/0123434 A1
(43) Pub. Date: May 14, 2009

(54) Cellular upregulation of VEGF receptor expression using nanofibrillar articles

(75) Inventor: Jeff J. Ross, Chaska, MN (US)

Correspondence Address: Kagan Binder, PLLC, 221 Main Street North, Suite 200, Stillwater, MN, 55082, US

(73) Assignee: SurModics, Inc.

(21) Appl. No.: 12/229,474

(22) Filed: Aug. 22, 2008

Related U.S. Application Data
(60) Provisional application No. 60/965,717, filed on Aug. 22, 2007.

(51) Int. Cl. A61K 35/12; C12N 5/06; C12Q 1/68; G01N 33/53

(52) U.S. Cl. 424/93.7; 435/375; 435/6; 435/7.2

(57) ABSTRACT

The invention provides methods and systems for selectively upregulating the expression of a VEGF receptor in a cell using a synthetic nanofibrillar article. Exemplary embodiments relate to the upregulation of the VEGF receptor Flk-1 in endothelial cells. Cells having the selectively-upregulated VEGF receptor can be used in processes such as cell culturing, tissue engineering, cellular therapy, and drug discovery.

### CROSS-REFERENCE TO RELATED APPLICATION

This application claims the benefit of U.S. Provisional Patent Application Ser. No. 60/965,717, filed Aug. 22, 2007, entitled CELLULAR UPREGULATION OF VEGF RECEPTOR EXPRESSION USING NANOFIBRILLAR ARTICLES, the disclosure of which is incorporated herein by reference.

### FIELD OF THE INVENTION

The invention relates to methods and systems using nanofibrillar articles for upregulating the expression of a vascular endothelial growth factor receptor (VEGF-R) in cells and tissue, as well as in vivo and in vitro uses of the VEGF-R upregulated cells and tissue.

### BACKGROUND

The tyrosine kinase receptor family includes a large number of transmembrane receptors that bind to polypeptide growth factors and have diverse biological activities. Their intrinsic tyrosine kinase function is activated upon ligand binding, which results in phosphorylation of the receptor and multiple cellular substrates, and subsequently in a variety of cellular responses (Ullrich A. and Schlessinger, J., 1990, Cell 61:203-212).

One class of receptor tyrosine kinases are those that bind to vascular endothelial growth factor (VEGF). VEFG is understood to be an important signaling protein involved in both vasculogenesis (the formation of the embryonic circulatory system) and angiogenesis (the growth of blood vessels from pre-existing vasculature).

VEGF acts mainly on cells of the vascular endothelium, but does have affects on a limited number of other cell types. In vitro, VEGF has been shown to stimulate endothelial cell mitogenesis and cell migration. VEGF also enhances microvascular permeability and is sometimes referred to as vascular permeability factor.

Vasculogenesis and angiogenesis play important roles in a variety of physiological processes such as embryonic development, wound healing, organ regeneration and female reproductive processes such as follicle development in the corpus luteum during ovulation and placental growth after pregnancy.

### SUMMARY

The present invention relates to methods and systems for selectively up-regulating the expression of a vascular endothelial growth factor (VEGF) receptor in cells or tissue using an article comprising a nanofibrillar structure. In some aspects of the invention, the methods and systems more specifically involve selectively up-regulating the VEGF receptor Flk-1.

The invention is based, in part, on experimental results showing a significant and selective upregulation of Flk-1 in endothelial cells in response to contact with an article comprising a nanofibrillar structure (“nanofibrillar article”). The increased expression of Flk-1 in endothelial cells following contacting the nanofibrillar article was much greater than the upregulation of Flk-1 expression in cells cultured on two-dimensional non-fibrillar plastic surfaces, or on Matrigel™-coated substrates.

Furthermore, and surprisingly, Flk-1 upregulation by the nanofibrillar article was highly selective as shown by the significant upregulation of Flk-1 as compared to the upregulation of expression other non-VEGF receptors on the endothelial cell surface. The increase in expression of Flk-1 was considerably greater than the increases seen in the non-VEGF receptors VE-cadherin, Tie-2, von Willebrand Factor receptor (vWR); and PECAM.

Desirably, following contact with the nanofibrillar article, the endothelial cells demonstrated reduced stress fibers with fiber alignment between multiple cells. Furthermore, the endothelial cells appeared to be multilayered without actin defined boarders. The phenotypic qualities, along with the strong selective upregulation of Flk-1, suggests that the endothelial cells contacted with the nanofibrillar article provide excellent material for cellular therapies. Exemplary therapies include those in which damaged tissue is treated to with the cells to promote an angiogenic and/or vasulogenic response.

Generally, the invention provides a method for selectively upregulating a VEGF receptor such as Flk-1 in cells or tissues The method comprises a step of contacting cells or tissue capable of upregulating a VEGF receptor with an article comprising a nanofibrillar structure. After the cells or tissue have been contacted, a significant and selective up-regulation of the VEGF receptor is observed in the cells or tissue. The step of contacting can be performed in vitro or in vivo. In some aspects the cells or tissue capable of upregulating a VEGF receptor are endothelial cells or tissue. In some aspects the VEGF receptor is Flk-1.

In some aspects, after the VEGF receptor has been upregulated, the method can comprise an additional step of contacting the cells with a compound that affects the VEGF receptor. In some cases, the compound can be a VEGF receptor agonist, such as VEGF or an analog thereof.

Various end uses for the cells are contemplated. For example, the cells having the upregulated VEGF receptor can be used therapeutically, or be used in an assay system, such as for the screening and discovery of compounds that affect the VEGF receptor.

The cells can be kept in contact with the nanofibrillar article for a period of time sufficient for upregulation of the VEGF receptor. In some aspects of the invention, the cells are kept in contact with the nanofibrillar article in vitro for a period of time of about 4 hours or greater, such as in the range of about 4 hours to about 30 days. In an exemplary mode of practice the cells are kept in contact with the nanofibrillar article for a period of about 20 hours.

The invention also provides systems comprising an article comprising a nanofibrillar structure and cells or tissue that are capable of selectively upregulating a VEGF receptor in response to the article, and/or cells or tissue that have selectively up-regulated a VEGF receptor in response to the nanofibrillar article.

The invention contemplates various implementations of the methods and systems of the invention, including, but not limited to, cellular therapies for the treatment of a medical condition in a subject, cell culturing methods, and diagnostic, screening, and analysis methods, such as methods for identifying compounds that affect a VEGF receptor, such as Flk-1.

Some aspects of the invention are directed to treating a medical condition in a subject using a cellular therapy. The contact with the nanofibrillar article can predispose the contacted cells to form tissue useful for the treatment of an individual.

The invention can be directed to cellular therapies directed at the formation of angiogenic or neovascular tissue. The specific upregulation of a VEGF receptor by the article comprising a nanofibrillar article provides an angiogenic marker on the contacted cells. Therefore, in another aspect, the invention provides a method for treating a subject comprising steps of (1) providing endothelial cells, (2) contacting cells or tissue with an article comprising a nanofibrillar structure in vitro, which provides contacted cells wherein the selective up-regulation of a VEGF receptor, such as Flk-1, has been promoted, and (3) implanting the contacted cells in a subject. The implanted cells can be used to treat a medical condition wherein the formation of new vascular tissue improves the medical condition. Examples of medical conditions that can be treated include those wherein body tissue has been damaged by lack of blood flow to the tissue (ischemia). Ischemic sites at which the contacted cells can be delivered to includes cardiac tissue, cerebral tissue, kidney tissue, lung tissue, and spleen tissue. Treatment using this cellular therapy can be carried out in a patient diagnosed with tachycardia, atherosclerosis, hypotension, or having suffered from a thromboembolism.

Another aspect of the invention involves the placement at a target site in the body an article comprising a nanofibrillar structure. In the method of the invention, the article comprising the nanofibrillar structure is implanted at a target site in the body. At the target site the nanofibrillar article is allowed to contact endothelial cells. Following contact, a VEGF receptor is specifically upregulated. The contacted cells can be used to treat a medical condition wherein the formation of new vascular tissue improves the medical condition, as described herein. In some cases the nanofibrillar article is a particulate comprising a nanofiber structure. In some cases the article comprises a biodegradable nanofiber structure. Following the step of contacting the endothelial cell the article can degrade in the body.

In some aspects of the invention, following the step of implanting the VEFG-R upregulated cells in the body, or after the cells have been upregulated by a nanofibrillar article placed in the body, a step of providing a therapeutic compound that affects the VEFG receptor can be performed.

A culturing process can also be carried out for the preparation of a sample of cells or tissue having increased expression of a VEGF receptor, wherein the cells or tissue are subsequently treated to obtain the VEGF receptor.

Another aspect of the invention involves the methods for screening test compounds or the discovery of bioactive agents. The compounds or bioactive agents can be tested to determine if they affect and/or interact with cells or tissue having increased expression of a VEGF receptor, such as Flk-1

In this aspect, the invention provides a method comprising steps of (1) providing a cell or tissue capable of upregulating a VEGF receptor, (2) contacting the cells or tissue with an article comprising a nanofibrillar structure, which promotes the selective up-regulation of the VEGF receptor in the cells or tissue, (3) applying a compound to the contacted cells, and (4) determining the effect of the compound on the contacted cells or the interaction of the compound with the VEGF receptor. In some aspects, in the step of providing, the cells are endothelial cells, and the VEGF receptor is Flk-1. The method can be performed in vitro or in vivo.

The method can be used determining if the compound has an affect on Flk-1 expressing cells, as opposed to cells wherein Flk-1 expression is not increased. Analysis can be performed to determine if the compound binds to Flk-1 (for example, if the compound is an agonist or antagonist). The screening of compounds using cells with selectively upregulated Flk-1 expression is desirable to identify compounds that can affect processes such as angiogenesis or vascularization.

Inhibiting angiogenesis is desirable in certain clinical situations (e.g., to suppress growth and mestastasis of solid tumors, or in treating rheumatoid arthritis), whereas promoting vascularization is beneficial for treating other conditions (e.g., wound healing). The present invention facilitates the discovery of molecules that promote angiogenesis by transducing signals through the above-discussed receptors, and molecules capable of inhibiting such signal transduction.

The invention also comprises a system comprising an article comprising a nanofibrillar substrate and endothelial cells, wherein the cells have been contacted with the article and demonstrate selective upregulation of Flk-1.

The methods and systems of the invention provide advantages in the areas of cellular therapies, cell culturing methods, and analysis methods, such as methods for screening or analyzing drugs. One advantage is that Flk-1 expression can be upregulated in endothelial cells without requiring exogenous substances. In some ways this provides a more natural way to promote formation of a desired cellular phenotype and accordingly eliminates or minimizes the carry over of an exogenous substance into a subject receiving the contacted cells.

### BRIEF DESCRIPTION OF THE DRAWINGS

FIGS. 1a-1h are fluorescence micrographic images of human umbilical vein endothelial cells (HUVEC) cells stained for actin with phalloidin following culturing on standard tissue culture surfaces (FIGS. 1a and 1b), biological tissue culture surfaces (FIGS. 1c, 1d, and 1e), Matrigel™-coated surface (FIG. 1f), and nanofiber surfaces (1g and 1h).

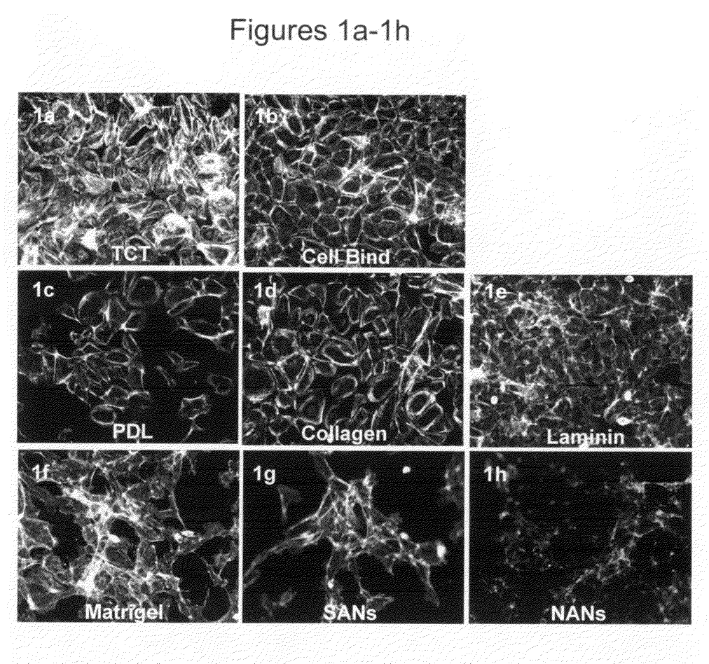

FIGS. 2 and 3 are graphs representing the increase in Flk-1 gene expression in aortic endothelial cells cultured on following culturing on standard tissue culture surfaces, Matrigel™-coated surface, and nanofiber surfaces at the fifth cell passage (FIG. 2) and the sixth cell passage (FIG. 3) measured relative to Flk-1 gene expression during the fourth cell passage.

FIGS. 4 and 5 are graphs representing the aortic endothelial cell population following culturing on standard tissue culture surfaces, Matrigel™-coated surface, and nanofiber surfaces at the third cell passage (FIG. 4) average cell doubling time on each of these surfaces (FIG. 5).

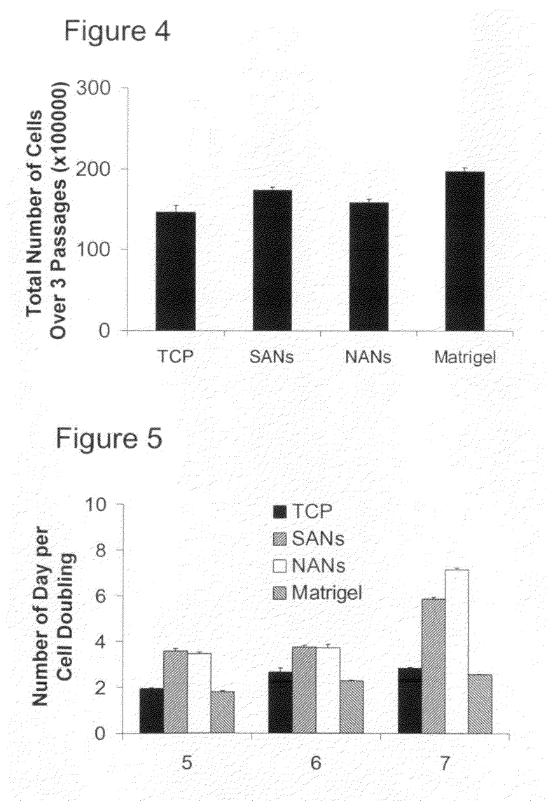

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention relates to methods and systems for selectively up-regulating the expression of receptors for vascular endothelial growth factor (VEGF) receptors in cells or tissue using an article comprising a nanofibrillar structure. In some aspects of the invention, the methods and systems involve selectively up-regulating Flk-1 using an article comprising a nanofibrillar structure.

Considerable information is available in the art regarding the biochemical and cellular functions, and genetic sequences of VEGF receptors (including Flk-1). This information can be used to identify available probes against VEGF receptors in order to determine their expression. For example, information regarding the genetic sequence of Flk-1 can be used to design nucleic acids as probes against the Flk-1 transcripts.

Various antibodies are commercially available against the Flk-1 protein (Santa Cruz Biotechnology, Inc., Santa Cruz, Calif.), as well as other VEGF receptors, including Flt-1, Flt-3, and Flt-4. These antibodies can be used to determine or confirm upregulation of the VEGF receptor on the cell surface in response to contact with the nanofiber substrate.

The receptors for vascular epithelial growth factor (VEGF) and related ligands include Flt-1 (VEGFR-1), Flk-1 (VEGFR-2), and Flt-4 (VEGFR-3), neuropilin-1, and neuropilin-2. These receptors have multiple immunoglobulin G-like extracellular domains and intracellular tyrosine kinase activity. There are several splice variants of VEGF, including VEGF 121, 145, 165, 189, and 206, with VEGF165 being the predominant form. Other members of the VEGF family have been cloned including VEGF-B, -C, and -D and placental growth factor (P1GF). VEGF binds to Flt-1 and Flk-1 and is involved in triggering angiogenesis. P1GF is localized to the placenta and binds only to Flt-1. VEGF-B also binds only to Flt-1 and appears to be involved in coronary vascularization and growth. VEGF-C and VEGF-D activate Flk-1 and Flt-4 but not VEGF-1. VEGF-C is implicated in lymphangiogenesis. (Yancopoulos, G. D., et al., Vascular-specific growth factors and blood vessel formation. Nature, 407, 242-248 (2000); Robinson, C. J., and Stringer, S. E., The splice variants of vascular endothelial growth factor (VEGF) and their receptors. J. Cell Sci., 114, 853-865 (2001)).

Flk-1 (Fetal liver kinase 1), is a member of the type III subclass of RTKs. An alternative designation for human Flk-1 is kinase insert domain-containing receptor (KDR) (Terman et al., Oncogene 6:1677-83, 1991), and the rat homolog has been termed TKr-C (Sarzani et al., Biochem. Biophys. Res. Comm. 186:706-714, 1992). DNAs encoding mouse, rat and human Flk-1 have been isolated, and the nucleotide and encoded amino acid sequences reported (Matthews et al., Proc. Natl. Acad. Sci. USA, 88:9026-30, 1991; Terman et al., 1991, supra; Terman et al., Biochem. Biophys. Res. Comm. 187:1579-86, 1992; Sarzani et al., supra; and Millauer et al., Cell 72:835-846, 1993). The type III subclass RTK designated fms-like tyrosine kinase-1 (Flt-1) is related to Flk-1 (DeVries et al., Science 255:989-991, 1992; Shibuya et al., Oncogene 5:519-524, 1990). Flt-1 is believed to be essential for endothelial organization during vascular development. Flt-1 expression is associated with early vascular development in mouse embryos, and with neovascularization during wound healing (Mustonen and Alitalo, J. Cell Biol. 1995 May; 129(4):895-8.). Expression of Flt-1 in adult organs suggests an additional function for this receptor that is not related to cell growth

Flt4 is related to Flt1 and Flk1 is Flt4 (Galland et al., Oncogene 8:1233-40, 1993; Pajusola et al., Oncogene 8:2931-37, 1993). The amino acid sequence of Flt-4 exhibits significant homology with the sequences of Flt1 and Flk-1, especially in the tyrosine kinase domain (Galland et al., supra). Unlike Flt-1 and Flk-1, however, a precursor form of Flt-4 is cleaved during post-translational processing to form two disulfide-linked polypeptides (Pajusola et al., supra). Studies of Flt-4 expression during development support the theory of venous origin of lymphatic vessels (Kaipainen et al., Proc. Natl. Acad. Sci. USA 92:3566-70, April, 1995).

Vascular endothelial cell growth factor (VEGF) binds to both Flk-1 and Flt-1 with relatively high affinity and is mitogenic toward vascular endothelial cells (Terman et al., 1992, supra; Mustonen et al. supra; DeVries et al., supra). VEGF and Flk-1 are a ligand-receptor pair thought to play an important role in the formation and sprouting of blood vessels, termed vasculogenesis and angiogenesis, respectively.

Cells or tissue in which a VEGF receptor can be selectively upregulated following contact with the nanofibrillar substrate are used in methods of the invention. In some aspects, a cell population comprising cells selected from the group consisting of primary cells, cells lines that are difficult to culture, and stem cells are placed in contact with the nanofiber article. Primary cell types include, mononuclear cells, stem or progenitor cells, lineage-specific cell types, marrow stromal cells, endothelial cells (including HUVEC), and leukemia and lymphoma cells. These cells can be obtained from sources such as peripheral blood, umbillical cord blood, and marrow. These cell types can be obtained from commercial sources (for example, Stem Cell Technologies, Inc., or Sigma-Aldrich) or prepared from a desired tissue using known techniques.

In one aspect of the invention, endothelial cells are placed in contact with the article comprising the nanofibrillar substrate. Endothelial cells are generally derived from the endothelium, which is the layer of thin specialized epithelium, comprising a simple squamous layer of cells that line the interior surface of blood vessels, forming an interface between circulating blood in the lumen and the rest of the vessel wall. Endothelial cells line the entire circulatory system, from the heart to the smallest capillary. Endothelial cells are very flat, have a central nucleus, are about 1-2 μm thick and about 10-20 μm in diameter. They form flat, pavement-like patterns on the inside of the vessels and at the junctions between cells there are overlapping regions which help to seal the vessel.

Endothelial cells may be derived from any part of the vascular tree. They may come from large and small veins and arteries, from capillaries, or from specialized vascular areas such as the umbilical vein of newborns, blood vessels in the brain or from vascularized solid tumors. Examples of tissue sources for endothelial cells include human umbilical vein or artery (HUVEC or HUAEC), human sephenous vein (HSVEC), human coronary artery (HCAEC), human pulmonary artery (HPAEC), human aorta (HAOEC), and foreskin, dermal tissue, or lung (Human microvascular endothelial cell; HMVEC).

Commercially available media have also been developed using that include endothelial cells.

The methods and systems of the invention involve contacting the cell or tissue capable of upregulating a VEGF receptor with an article comprising a nanofibrillar structure.

A “nanofibrillar structure” refers to a mesh-like network of nanofibers. The article comprising the nanofibrillar structure can be formed of the nanofibrillar structure alone or the nanofibrillar structure along with a non-nanofibrillar portion.

A nanofibrillar structure can be a cell culture article and can be included in any sort of cell culture apparatus wherein cell attachment is desired, or where a cell culture process is performed. In many cases an article that includes a network of nanofibers includes a network of nanofibers in addition to one or more other non-nanofiber materials. For example, a nanofibrillar structure can include a network of nanofibers on a support, wherein the support is fabricated from a material that is different than the nanofibers. A nanofibrillar structure can also be used with articles that are not used in in vitro cell culture processes.

A “cell culture article” refers to any portion of a cell culture apparatus. For example, a cell culture article can be an article having a nanofibrillar structure. A cell culture article can also be a receptacle used in a cell culture process, such as a cell culture vessel. In some cases two or more cell culture articles (such as a nanofibrillar structure and a cell culture container) form a cell culture apparatus; in other cases a single cell culture article, such as the nanofibrillar structure, constitutes a cell culture apparatus.

The nanofibrillar structure can be placed on the surface of another cell culture article, such as a cell culture vessel. In many aspects, the nanofibrillar structure can be “adapted for insertion” into another article. This means that the nanofibrillar structure can be manufactured or fabricated for use in, or to the dimensions of one or more surfaces of another article, such as a cell culture vessel. The nanofibrillar structure can be sized for use in or to the dimensions of a surface of the culture vessel by, for example, cutting down the nanofibrillar structure to a particular size, for example cutting a piece of the nanofibrillar structure from an associated sheet, roll, or mat, to a size suitable for insertion onto a surface of the culture vessel.

A “cell culture vessel” is an example of a cell culture article and, as used herein, means a receptacle that can be associated with the nanofibrillar structure and can contain media for culturing a cell or tissue. The cell culture vessel may be glass or plastic. Preferably the plastic is non-cytotoxic. Exemplary cell culture vessels include, but are not limited to, single and multi-well plates, including 6 well and 12 well culture plates, and smaller welled culture plates such as 96, 384, and 1536 well plates, culture jars, culture dishes, petri dishes, culture flasks, culture plates, culture roller bottles, culture slides, including chambered and multi-chambered culture slides, culture tubes, coverslips, cups, spinner bottles, perfusion chambers, bioreactors, and fermenters.

A nanofibrillar structure can be in the form of a “mat” which as used herein means a densely interwoven, tangled, or adhered mass of nanofibers. The distribution of nanofibers in the mat may be random or oriented. A mat may be unwoven or net. A mat may or may not be deposited on a support. A mat has a thickness of about 100 nm to about 10,000 nm, or about 1000 nm to about 5000 nm.

Depending on the method of fabrication and the types of cells that are cultured, cells can grow in one plane, or more than one plane on the nanofibrillar structure. Generally, the coated surfaces of cell culture articles that include a nanofibrillar structure resemble scaffoldings on which cells can attach.

The materials used to form the nanofibrillar structure are referred to herein as “nanofiber materials” whereas. Other materials can be associated with nanofibers, such as coating materials.

Exemplary nanofibrillar structures are described in U.S. Patent Pub. No. 2005/0095695A1.

The nanofibrillar structure provides an environment for the culturing of metabolically active cells comprising one or more nanofibers, wherein the structure is defined by a network of one or more nanofibers. In some embodiments, the nanofibrillar structure comprises a substrate wherein the nanofibrillar structure is defined by a network of one or more nanofibers deposited on a surface of the substrate. The nanotopography, the topography of the nanofiber network and the arrangement of the nanofibers of the nanofiber network in space, of the nanofibrillar structure is engineered to provide an in vitro biomimetic substratum that is tissue compatible for the promotion of homotypic or heterotypic cell growth and/or cell differentiation in single layer or multi-layered cell culture. The nanofibrillar structures can be layered to form a multi-layered nanofibrillar assembly, cellular array, or tissue structure.

The term “network” as used herein means a random or oriented distribution of nanofibers in space that is controlled to form an interconnecting net with spacing between fibers selected to promote growth and culture stability. The network has small spaces between the fibers comprising the network forming pores or channels in the network. The pores or channels have a diameter of about 0.01 microns to about 25 microns, and more typically about 0.2 microns to about 10 microns, through a thickness. Advantageously, the polymeric coatings that are formed on the nanofibers do not significantly reduce the diameter of the pores or channels.

A network may comprise a single layer of nanofibers, a single layer formed by a continuous nanofiber, multiple layers of nanofibers, multiple layers formed by a continuous nanofiber, or mat. The network may be unwoven or net. A network may have a thickness of about the diameter of a single nanofiber to about 10 μm. Physical properties of the network including, but not limited to, texture, rugosity, adhesivity, porosity, solidity, elasticity, geometry, interconnectivity, surface to volume ratio, fiber diameter, fiber solubility/insolubility, hydrophilicity/hydrophobicity, fibril density, and fiber orientation may be engineered to desired parameters. A polymeric coating can be formed on the nanofibers without significantly changing the beneficial properties provided by the network of nanofibers. For example, the polymeric coating does not alter the structural features of the nanofibrillar structure in such a way as that it reduces its function as a biomimetic substratum.

The term “nanofiber” as used herein means a polymer fine fiber comprising a diameter of about 1000 nanometers or less.

A wide range of polymeric materials can be used as nanofiber materials in the preparation of the nanofibrillar structures. Nanofiber materials can include both addition polymer and condensation polymer materials such as polyolefin, polyacetal, polyamide, polyester, cellulose ether and ester, polyalkylene sulfide, polyarylene oxide, polysulfone, modified polysulfone polymers and mixtures thereof. Exemplary materials within these generic classes include polyethylene, poly(ε-caprolactone), poly(lactate), poly(glycolate), polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms. Exemplary addition polymers tend to be glassy (a Tg greater than room temperature). This is the case for polyvinylchloride and polymethylmethacrylate, polystyrene polymer compositions, or alloys or low in crystallinity for polyvinylidene fluoride and polyvinyl alcohol materials.

In some cases the nanofiber material is a polyamide condensation polymer. In more specific embodiments, the polyamide condensation polymer is a nylon polymer. The term “nylon” is a generic name for all long chain synthetic polyamides. Typically, nylon nomenclature includes a series of numbers such as in nylon-6,6 which indicates that the starting materials are a C₆ diamine and a C₆ diacid (the first digit indicating a C₆ diamine and the second digit indicating a C₆ dicarboxylic acid compound). Another nylon can be made by the polycondensation of epsilon caprolactam in the presence of a small amount of water. This reaction forms a nylon-6 (made from a cyclic lactam—also known as epsilon-aminocaproic acid) that is a linear polyamide. Further, nylon copolymers are also contemplated. Copolymers can be made by combining various diamine compounds, various diacid compounds and various cyclic lactam structures in a reaction mixture and then forming the nylon with randomly positioned monomeric materials in a polyamide structure. For example, a nylon 6,6-6,10 material is a nylon manufactured from hexamethylene diamine and a C₆ and a C₁₀ blend of diacids. A nylon 6-6,6-6,10 is a nylon manufactured by copolymerization of epsilon aminocaproic acid, hexamethylene diamine and a blend of a C₆ and a C₁₀ diacid material.

Block copolymers can also be used as nanofiber materials. In preparing a composition for the preparation of nanofibers, a solvent system can be chosen such that both blocks are soluble in the solvent. One example is an ABA (styrene-EP-styrene) or AB (styrene-EP) polymer in methylene chloride solvent. Examples of such block copolymers are Kraton™ type of AB and ABA block polymers including styrene/butadiene and styrene/hydrogenated butadiene(ethylene propylene), Pebax™ type of epsilon-caprolactam/ethylene oxide, Sympatex™ polyester/ethylene oxide and polyurethanes of ethylene oxide and isocyanates.

Addition polymers such as polyvinylidene fluoride, syndiotactic polystyrene, copolymers of vinylidene fluoride and hexafluoropropylene, polyvinyl alcohol, polyvinyl acetate, amorphous addition polymers, such as poly(acrylonitrile) and its copolymers with acrylic acid and methacrylates, polystyrene, poly(vinyl chloride) and its various copolymers, poly(methyl methacrylate) and its various copolymers, can be solution spun with relative ease because they are soluble at low pressures and temperatures. Highly crystalline polymer like polyethylene and polypropylene generally require higher temperature and high pressure solvents if they are to be solution spun. Electrostatic solution spinning is one method of making nanofibers and microfiber.

Nanofibers can also be formed from polymeric compositions comprising two or more polymeric materials in polymer admixture, alloy format, or in a crosslinked chemically bonded structure. Such polymer compositions can physical properties by changing polymer attributes such as improving polymer chain flexibility or chain mobility, increasing overall molecular weight and providing reinforcement through the formation of networks of polymeric materials.

Two related polymer materials can be blended to provide the nanofiber with beneficial properties. For example, a high molecular weight polyvinylchloride can be blended with a low molecular weight polyvinylchloride. Similarly, a high molecular weight nylon material can be blended with a low molecular weight nylon material. Further, differing species of a general polymeric genus can be blended. For example, a high molecular weight styrene material can be blended with a low molecular weight, high impact polystyrene. A Nylon-6 material can be blended with a nylon copolymer such as a Nylon-6; 6,6; 6,10 copolymer. Further, a polyvinyl alcohol having a low degree of hydrolysis such as a 87% hydrolyzed polyvinyl alcohol can be blended with a fully or super hydrolyzed polyvinyl alcohol having a degree of hydrolysis between 98 and 99.9% and higher. All of these materials in admixture can be crosslinked using appropriate crosslinking mechanisms. Nylons can be crosslinked using crosslinking agents that are reactive with the nitrogen atom in the amide linkage. Polyvinyl alcohol materials can be crosslinked using hydroxyl reactive materials such as monoaldehydes, such as formaldehyde, ureas, melamine-formaldehyde resin and its analogues, boric acids, and other inorganic compounds, dialdehydes, diacids, urethanes, epoxies, and other known crosslinking agents. Crosslinking reagent reacts and forms covalent bonds between polymer chains to substantially improve molecular weight, chemical resistance, overall strength and resistance to mechanical degradation.

Electrospinning produces a population of nanofibers that may differ in diameter, typically from about 5 nm to about 1000 nm.

Nanofibers can be produced by the electrospinning process that uses an electric field to control the formation and deposition of polymers. A polymer solution is injected with an electrical potential. The electrical potential creates a charge imbalance that leads to the ejection of a polymer solution stream from the tip of an emitter such as a needle. The polymer jet within the electric field is directed toward a grounded substrate, during which time the solvent evaporates and fibers are formed. The resulting single continuous filament collects as a nonwoven fabric on a support. Electrospinning processes for the production of nanofibers have been described in U.S. Pat. Nos. 4,650,506 (Barris) and 6,743,273 (Chung et al.). A nanofiber structure being manmade can be referred to as a synthetic nanofiber structure, and can include synthetic/manmade materials, natural materials, or both.

Electrospun nanofiber networks may be produced having random or directed orientations. Random fibers may be assembled into layered surfaces. In some embodiments, the nanofibers comprise a random distribution of fine fibers that can be bonded to form an interlocking network. The nanofiber interlocking networks have relatively small spaces between the fibers. Such spaces typically range, between fibers, of about 0.01 to about 25 microns, preferably about 2 to about 10 microns. Such spaces form pores or channels in the nanofiber network allowing for diffusion of ions, metabolites, proteins, and/or bioactive molecules and/or allowing cells to penetrate and permeate the network and grow in an environment that promotes multipoint attachments between cells and the nanofibers.

Nanofiber networks may also be electrospun in an oriented manner. Such oriented electrospinning allows for the fabrication of a nanofiber network comprising a single layer of nanofibers or a single layer formed by a continuous nanofiber wherein the network has a height of the diameter of a single nanofiber. Physical properties including porosity, solidity, fibril density, texture, rugosity, and fiber orientation of the single layer network may be selected by controlling the direction and/or orientation of the nanofiber onto the support during the electrospinning process. Preferably the pore size allows cells to penetrate and/or migrate through the single layer nanofiber network. In an embodiment, the space between fibers is about 0.01 to about 25 microns. In another embodiment, the space between fibers is about 2 to about 10 microns.

The nanofiber may optionally include other materials.

In some aspects, the nanofibers can be formed by combining a polymeric material with an additive composition. The additive composition can influence packing of the polymer such that electrospinning of the polymer results in the production of a population of nanofibers having a greater number or percentage of thin fibers as compared to a population of nanofibers electrospun form a polymer solution not containing the additive. In an embodiment, the polymer solution comprises from about 0.25% to about 15% w/w additive composition. In another embodiment, the polymer solution comprises from about 1% to about 10% w/w additive composition. In some embodiments, the additive composition that influences packing of the polymer includes a bioactive molecule such as a lipid. In some embodiments the lipid can be selected from the group consisting of lysophosphatidylcholine, phosphatidyl-choline, sphingomyelin, cholesterol, and mixtures thereof.

The nanofibrillar structure may be comprised of multiple nanofibers having different diameters and/or multiple nanofibers fabricated from different polymers. Solidity of the nanofibrillar structure may also be engineered to affect cell growth and/or differentiation. In an embodiment, the nanofibrillar structure has a solidity of about 3 percent to about 70 percent. In another embodiment, the nanofibrillar structure has a solidity of about 3 percent to about 50 percent. In another embodiment, the nanofibrillar structure has a solidity of about 3 percent to about 30 percent. In another embodiment, the nanofibrillar structure has a solidity of about 3 percent to about 10 percent. In another embodiment, the nanofibrillar structure has a solidity of about 3 percent to about 5 percent. In another embodiment, the nanofibrillar structure has a solidity of about 10 percent to about 30 percent.

In some aspects the nanofibers include a fluorescent marker. The fluorescent marker allows, for example, visualization of a nanofiber, identification of specific nanofibers within a nanofiber blend, identification of a chemical or physical property of a nanofiber, and evaluation of the degradation of and/or redistribution of nanofibers and/or structures comprising nanofibers, including multi-layered assemblies useful for engineering tissue. The fluorescent marker may be photobleachable or non-photobleachable. The fluorescent marker may be pH sensitive or pH insensitive. Preferably the fluorescent marker is non-cytotoxic.

The polymers used to form the nanofibers can also have adhering characteristic such that when contacted with a cellulosic, polyvinyl, polyester, polystyrene, or polyamide support, the nanofiber adheres to the support with sufficient strength such that it is securely bonded to the support and can resist delaminating effects associated with mechanical stresses. Adhesion of the nanofiber to the support can arise from solvent effects of fiber formation as the fiber is contacted with the support or the post treatment of the fiber on the support with heat or pressure. Polymers plasticized with solvent or steam at the time of adhesion can have increased adhesion.

The term “nanofibrillar support” as used herein means any surface on which nanofiber or network of nanofibers is deposited. The nanofibrillar support may be any surface that offers structural support for the deposited network of nanofibers. The nanofibrillar support may comprise glass or plastic. Preferably the plastic is non-cytotoxic. In some aspects, the nanofibrillar support may be a film or culture container.

The nanofibrillar support may be water-soluble or water insoluble. A nanofibrillar support that is water-soluble is preferably a polyvinyl alcohol film. In many aspects, and for most methods, the average size of the pores in the nanofibrillar structure is too small to allow for cell entry into the nanofibrillar structure. However, the movement of cells may depend on the size of the cell and the size of the pores in the nanofibrillar structure.

Preferably the pores in a porous nanofibrillar structure have a diameter of about 0.2 tam to about 10 μm. The nanofibrillar structure may be biodegradable and/or biodissolvable. Preferably the nanofibrillar structure is biocompatible.

In some aspects, the nanofibrillar support is selected from glass substrates, such as glass microscope slides, glass cover slips, and glass films; polymer substrates, such as polymer films; and other biomaterial substrates suitable for cell culturing. The nanofibrillar support can have certain properties useful for the useful for the preparation and/or use of a cell culture device. In some specific aspects the nanofibrillar support has one or more properties such as manipulability and/or flexibility, resistance to sterilization, resistance to degradation by radiation, chemical inertness, transparency, non-flammability, and smooth surface properties. In some specific aspects, the nanofibrillar support includes a halogenated thermoplastic resin, such as halogenated fluorinated-chlorinated resins. Specifically, the support can include chlorotrifluoroethylene (CTFE). In other specific aspects, the support is about 0.25 mm or less.

The term “spacer” as used herein means a layer separating a nanofiber or nanofiber network from a surface of a support or a surface of another nanofibrillar structure such that the structures are separated by the diameter or thickness of the spacer. The spacer may comprise a polymer fine fiber or film. Preferably the film has a thickness of about 10 microns to about 50 microns. The spacer may comprise a polymer including cellulose, starch, polyamide, polyester, or polytetrafluoroethylene. The fine fiber may comprise a microfiber. A microfiber is a polymer fine fiber comprising a diameter of about 1 μm to about 30 μm. The microfiber may be unwoven or net.

The nanofibrillar structure can be associated with or formed on articles such as supports, cell culture articles, and medical devices. Such articles can be combined or fabricated with the nanofibrillar structure to form various articles or assemblies, including cell culture apparatuses and medical devices. Articles such as supports, cell culture articles, and medical devices can be made of the same material as the nanofibrillar structure, or can be made from different materials.

Example of materials which can be used to form an article associated with the nanofibrillar structure, such as a support, cell culture article, or medical device, include synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, and styrene.

Exemplary polymeric materials commonly used in cell culture articles include polystyrene and polypropylene.

Biodegradable polymers can also be used in the preparation of an article associated with the nanofibrillar structure. Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with, for example, implantable medical devices include polylactide, polygylcolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used.

The nanofibrillar article can also include a coating. The coating can be formed from materials comprising non-biodegradable or degradable polymers. The experimental results described herein also show that using an article comprising coated nanofibers can be particularly beneficial for promoting the upregulation of Flk-1. As shown in FIG. 3, at a later time point in cell culturing, the coated nanofiber substrate promoted the greatest upregulation of Flk-1 (compared to the uncoated nanofiber substrate, and the Matrigel™ and tissue culture plastic substrates). Remarkably, the upregulation of the non-VEGF receptors was minimal.

Exemplary nanofibrillar coatings are described in U.S. Publication No. 2007/0082393 (Lodhi et al.)

In some aspects, the nanofibers of the nanofibrillar structure have a coating that includes a non-biodegradable polymer one or more and preferably a plurality of pendent amine groups, and one or preferably more than one pendent latent reactive groups. “Non-biodegradable” refers to polymers that are generally not able to be non-enzymatically, hydrolytically or enzymatically degraded. For example, the non-biodegradable polymer is resistant to degradation that may be caused by proteases.

The coating on the nanofibers can be formed through use of latent reactive groups wherein at least a portion of the groups are activated during the coating process to bond the polymer to the surface of the nanofibers of the nanofibrillar structure. The polymer (in the formed coating) that is covalently bonded to the surface of the nanofibers may be referred to as having “latent reacted groups,” or, “reacted groups,” referring to one or more of these latent reactive groups on the polymer has been activated and reacted to form a covalent bond between the polymer and the surface of the nanofiber.

By binding to the surface via the latent reacted groups, the immobilized polymer provides positively-charged amine groups to the surface of the nanofibers of the nanofibrillar structure. It is thought that this binding arrangement allows for the formation of a very durable and effective coating.

The plurality of pendent amine groups on the non-biodegradable polymer can provide a positive charge to the coating in pH conditions suitable for cell culture. For example, the non-biodegradable polymer will provide a positive charge to the nanofibers of the nanofibrillar structure in conditions ranging from about pH 5.0 to about pH 10.0.

The non-biodegradable polymer can have primary amine, secondary amine, tertiary amine, or combinations of these amine groups pendent from the polymer.

In one aspect, an exemplary amine-containing group has the following formula: R₁R₂NR₃R₄, wherein R₁ is:

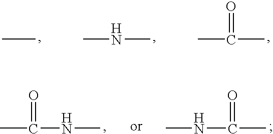

wherein R₂ is C₁-C₈ linear or branched alkyl; and wherein R₃ and R₄ are both attached to the nitrogen and are individually H or C₁-C₆ linear or branched alkyl. As pendent from the polymer, the amine-containing group can be represented by the formula P—[R₁R₂NR₃R₄], P being a portion of the polymeric backbone.

In some more specific aspects, R₁ is

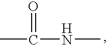

R₂ is C₂-C₄ linear or branched alkyl; and, R₃ and R₄ are both attached to the nitrogen and are individually H, CH₃, or C₂H₅.

Exemplary amine containing groups include those found on polymerizable monomers such as 3-aminopropylmethacrylamide (APMA), 3-aminoethylmethacrylamide (AEMA), dimethylaminopropylmethacrylamide (DMAPMA), and the like. Therefore, in some aspects, polymers including pendent amine groups and latent reactive groups can be formed by copolymerizing a monomer having a group —R₁R₂NR₃R₄ as defined above with a comonomer bearing a latent reactive group. Optionally, other non-amine or non-latent reactive group-containing monomers can be included in the polymer.

In one aspect, the polymer includes an amine-containing group in a molar amount of about 10% or greater, as based on the monomer content of the polymer. This can be achieved, for example, by preparing a polymer with 10% or greater of an amine-containing group monomer. In some aspects, the amine-containing group is present in a molar amount of 50% or greater, 60% or greater, 70% or greater, 80% or greater, 90% or greater, or 95% or greater. In some aspects the polymer includes amine-containing groups in a molar amount in the range of about 90%-99.95%. An exemplary preparation of a copolymer includes about 98.4% amine-containing monomer, such as APMA, AEMA, or DMAPMA and about 1.6% of monomer including the latent reactive group.

Control over amount of amine group and amount of latent reactive group can be exercised by copolymerizing an amine-containing monomer with a latent reactive group-containing monomer (and optionally a non-amine or non-photo reactive group-containing monomer). Other exemplary amine-containing polymers can be formed by the copolymerization of, for example, amine-containing monomers such as N-(2-amino-2-methylpropyl)methacrylamide, p-aminostyrene, allyl amine, or combinations thereof with a monomer having a pendent latent reactive groups to provide a polymer having pendent amine groups and latent reactive groups. These amine-containing monomers can also be copolymerized with other non-primary amine-containing monomers, such as acrylamide, methacrylamide, vinyl pyrrolidinone, or derivatives thereof, to provide a polymer having desired properties, such as a desired density of amine groups and photoreactive groups. Other suitable polymers that have amine groups include polymers that are formed from monomers such as 2-aminomethylmethacrylate, 3-(aminopropyl)-methacrylamide, and diallylamine. Dendrimers that include photogroups and pendent amine groups can also be used.

In some aspects a polymer having pendent amine groups and hydrophobic properties can be prepared. This can be achieved by one or more schemes for the synthesis of the polymer. For example, a polymer can be formed with a desired amount of hydrophobic monomers, such as (alkyl)acrylate monomers, or the amine-presenting monomers can include longer alkyl chain lengths. For example, any one or more of the groups R₂, R₃, and/or R₄ can include alkyl groups of 3 or more carbon atoms.

Another method for preparing the non-biodegradable polymer includes steps of derivatizing a preformed polymer with a compound that includes a latent reactive group. For example, a homopolymer or heteropolymer having pendent amine groups can be readily derivatized with a photoreactive group by reacting a portion of the pendent amine groups with a compound having a photoreactive group and a group that is reactive with an amine group, such as 4-benzoylbenzoyl chloride.

In some aspects, the polymer having pendent amine groups and at least one latent reactive group is selected from polyethyleneimine, polypropyleneimine, and polyamidoamine. In one specific aspect the polymeric material includes polyethyleneimine with one or more latent reactive group(s).

Latent reactive groups, broadly defined, are groups that respond to specific applied external stimuli to undergo active specie generation with resultant covalent bonding to a target. Latent reactive groups are those groups of atoms in a molecule that retain their covalent bonds unchanged under conditions of storage but which, upon activation, form covalent bonds with other molecules. The latent reactive groups generate active species such as free radicals, nitrenes, carbenes, and excited states of ketones upon absorption of external electromagnetic or kinetic (thermal) energy. Latent reactive groups may be chosen to be responsive to various portions of the electromagnetic spectrum, and latent reactive groups that are responsive to ultraviolet, visible or infrared portions of the spectrum are preferred. Latent reactive groups, including those that are described herein, are well known in the art. See, for example, U.S. Pat. No. 5,002,582 (Guire et al., “Preparation of Polymeric Surfaces Via Covalently Attaching Polymers”). Any suitable latent reactive group can be used for formation of a coating on the nanofibers.

Photoreactive groups can generate active species such as free radicals and particularly nitrenes, carbenes, and excited states of ketones, upon absorption of electromagnetic energy. Photoreactive groups can be chosen to be responsive to various portions of the electromagnetic spectrum, and that are responsive to the ultraviolet and visible portions of the spectrum are preferred.

Photoreactive aryl ketones are preferred, such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives. Examples of preferred aryl ketones include heterocyclic derivatives of anthrone, including acridone, xanthone, and thioxanthone, and their ring substituted derivatives. Some preferred photoreactive groups are thioxanthone, and its derivatives, having excitation energies greater than about 360 nm.

The functional groups of such ketones are preferred since they are readily capable of undergoing the activation/inactivation/reactivation cycle described herein. Benzophenone is a particularly preferred latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatable aryl ketones such as benzophenone and acetophenone are of particular importance inasmuch as these groups are subject to multiple reactivation in water and hence provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benezensulfonyl azide; and phosphoryl azides [(RO)₂PON₃] such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetatoacetates (—CO_CN₂CO—O—) such as t-butyl alpha diazoacetoacetate.

Other photoreactive groups include the diazirines (CHN₂) such as 3-trifluoromethyl-3-phenyldiazirine; and ketenes (CH═C═O) such as ketene and diphenylketene.

Peroxy compounds are contemplated as another class of latent reactive groups and include dialkyl peroxides such as di-t-butyl peroxide and dicyclohexyl peroxide and diacyl peroxides such as dibenzoyl peroxide and diacetyl peroxide and peroxyesters such as ethyl peroxybenzoate.

In some aspects, the latent reactive group is present in a molar amount (relative to the monomers of the polymer) of up to about 10%, or an amount of up to about 5%. In some aspects, the polymer includes the latent reactive group in a molar amount the range of about 0.05%-10%. An exemplary preparation of a copolymer includes about 98.4% amine-containing monomer, such as APMA, AEMA, or DMAPMA and about 1.6% of monomer including the latent reactive group.

As described above, a polymer having pendent amine groups and pendent latent reactive groups can be disposed on the surface of the nanofibers and the surface can be treated to activate the latent reactive groups thereby bonding the polymer to the surface of the nanofibers, and forming a thin polymeric coating over the nanofiber surface.

In another method, the polymer is formed on the nanofiber surface of the nanofibrillar structure by a graft polymerization method. For example, a monomer including a latent reactive group and a polymerizable group can be disposed and bonded to the surface of the nanofibers. A composition of monomers including amine groups can then be disposed on the surface, and a polymerization reaction can be initiated to cause the formation of a polymer chain from and bonded to the surface of the nanofibrillar structure.

In yet another method, the coating can be formed using a crosslinking agent having two or more latent reactive groups, wherein the crosslinking agent is used to bond the polymer to the surface of the nanofibers of the nanofibrillar structure. The crosslinking agent can have any two or more of the latent reactive groups as described herein. In forming the polymeric coating, the crosslinking agent can be disposed on the surface of the nanofibrillar structure followed by disposing the polymer having pendent amine groups, or the crosslinking agent can be disposed in combination with the polymer, or both.

If photoreactive groups are present on the cross-linking agent, preferably they are adapted to undergo reversible photolytic homolysis, thereby permitting photoreactive groups that are not consumed in attachment to a polymeric material to revert to an inactive, or “latent” state. These photoreactive groups can be subsequently activated, in order to attach to the polymer with an abstractable hydrogen for covalent bond formation. Thus, excitation of the photo reactive group is reversible and the group can return to a ground state energy level upon removal of the energy source. In some embodiments, preferred cross-linking agents are those groups that can be subject to multiple activations and hence provide increased coating efficiency. Exemplary crosslinking agents are described in Applicant's U.S. Pat. No. 5,414,075 (Swan et al.), and U.S. Publication No. 2003/0165613 A1 (Chappa et al.). See also U.S. Pat. Nos. 5,714,360 (Swan et al.) and 5,637,460 (Swan et al.).

The non-biodegradable polymer having pendent amine groups can be bonded to the nanofibers of a nanofibrillar structure either alone or with other optional components. In its simplest form, the coating composition consists of, for example, (i) a non-biodegradable polymer having at least one, or preferably a plurality of pendent amine groups, and least one latent reactive group and/or (ii) a non-biodegradable polymer having at least one, or preferably a plurality of pendent amine groups and a crosslinking agent having two or more or more latent reactive groups. Other components may be added to the coating composition to change or improve aspects of the coating. The components may be polymeric or non-polymeric components.

Other synthetic or natural, biodegradable or non-biodegradable polymers can be added to the composition to form the coating. A “synthetic polymer” refers to a polymer that is synthetically prepared and that includes non-naturally occurring monomeric units. For example, a synthetic polymer can include non-natural monomeric units such as acrylate, acrylamide, etc. Synthetic polymers are typically formed by traditional polymerization reactions, such as addition, condensation, or free-radical polymerizations. Synthetic polymers can also include those having natural monomeric units, such as naturally-occurring peptide, nucleotide, and saccharide monomeric units in combination with non-natural monomeric units (for example synthetic peptide, nucleotide, and saccharide derivatives). These types of synthetic polymers can be produced by standard synthetic techniques, such as by solid phase synthesis, or recombinantly, when allowed.

A “natural polymer” refers to a polymer that is either naturally, recombinantly, or synthetically prepared and that consists of naturally occurring monomeric units in the polymeric backbone. In some cases, the natural polymer may be modified, processed, derivatized, or otherwise treated to change the chemical and/or physical properties of the natural polymer. In these instances, the term “natural polymer” will be modified to reflect the change to the natural polymer (for example, a “derivatized natural polymer”, or a “deglycosylated natural polymer”).

Biodegradable materials, such as biodegradable polymers, can also be present in the coating. The biodegradable materials can optionally be present in the same coated layer as the non-biodegradable amine-presenting polymer, can be present in another coated layer, if included in the coating, or both. For example, a coated layer that includes a biodegradable polymer can be formed between the coated layer that includes the non-biodegradable amine-presenting polymer and the article surface, or can be formed on top of the coated layer that includes the non-biodegradable amine-presenting polymer. During culturing, the biodegradable polymer can degrade while the non-biodegradable polymer remains present in the coating and provides an adherent surface during protracted periods of culturing.

A “bioactive molecule” can be associated with the polymer-coated nanofibrillar structure. For example, one or more bioactive molecules can be present in the nanofiber, and/or in the coating on the nanofibers that contains the non-biodegradable polymer having pendent amine groups and latent reactive groups. In some cases the bioactive molecule can be a biodegradable material, as described herein. While one or more bioactive molecule(s) can be associated with the polymer-coated nanofibrillar structure, bioactive molecule(s) may also be included in liquid media when cell culture methods are performed in conjunction with the polymer-coated nanofibrillar structure. Therefore, recitation of bioactive molecules is not intended to limit the presence of the molecule to the coating or to any media, unless specifically described herein.

The term “bioactive molecule” as used herein means a molecule that has an effect on a cell or tissue. The term includes human or veterinary therapeutics, nutraceuticals, vitamins, salts, electrolytes, amino acids, peptides, polypeptides, proteins, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chitosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, IL-11, IL-12, IL-13, TNF-alpha, and TNF-beta. Immunoglobulins include, but are not limited to, IgG, IgA, IgM, IgD, IgE, and mixtures thereof. Amino acids, peptides, polypeptides, and proteins may include any type of such molecules of any size and complexity as well as combinations of such molecules. Examples include, but are not limited to, structural proteins, enzymes, and peptide hormones.

The term bioactive molecule also includes fibrous proteins, adhesion proteins, adhesive compounds, deadhesive compounds and targeting compounds. Fibrous proteins include collagen and elastin. Adhesion/deadhesion compounds include fibronectin, laminin, thrombospondin and tenascin C. Adhesive proteins include actin, fibrin, fibrinogen, fibronectin, vitronectin, laminin, cadherins, selectins, intracellular adhesion molecules 1, 2, and 3, and cell-matrix adhesion receptors including but not limited to integrins such as α₅β₁, α₆β₁, α₇β₁, α₄β₂, α₂β₃, α₆β₄.

Polymers that have traditionally been used to form coatings for cell attachment can be included in the coating composition. For example, polypeptide-based polymers such as polylysine, collagen, fibronectin, integrin, and laminin can be included in the coatings. Peptide portions of these polypeptides can also be included in the coating composition. Exemplary binding domain sequences of matrix proteins are shown in Table 1.

TABLE 1

| | | | |
|---|---|---|---|
| Fibronectin: | RGDS | LDV | REDV |
| Vitronectin | RGDV | | |
| Laminin A | LRGDN | IKVAV | |
| Laminin B1 | YIGSR | PDSGR | |
| Laminin B2 | RNIAEIIKDA | | |
| Collagen I | RGDT | DGEA | GTPGPQGIAGQRGVV |
| Thrombospondin | RGD | VTXG | FYVVMWK |

These polypeptide-based polymers can be in an underivatized or derivatized form. For example, the polypeptide-based polymers can be derivatized with latent reactive groups, and then can be activated along with the latent reactive groups pendent from the non-biodegradable polymer to form the coating. Exemplary combinations can include photo-poly(aminopropylmethacrylamide) or photo-poly(ethyleneimine) with one or more of photo-polylysine, photo-collagen, photo-fibronectin, and photo-laminin, or photo-derivitized portions of polypeptides, including those described herein.

Photoderivatized polypeptides, such as collagen, fibronectin, and laminin can be prepared as described in U.S. Pat. No. 5,744,515 (Clapper, Method and Implantable Article for Promoting Endothelialization). As described in this patent, a heterobi-functional agent can be used to photoderivatize a protein. The agent includes a benzophenone photoactivatable group on one end (benzoyl benzoic acid, BBA), a spacer in the middle (epsilon aminocaproic acid, EAC), and an amine reactive thermochemical coupling group on the other end (N-oxysuccinimide, NOS). BBA-EAC is synthesized from 4-benzoylbenzoyl chloride and 6-aminocaproic acid. Then the NOS ester of BBA-EAC is synthesized by esterifying the carboxy group of BBA-EAC by carbodiimide activation with N-hydroxysuccimide to yield BBA-EAC-NOS. Proteins, such as collagen, fibronectin, laminin, and the like can be obtained from commercial sources. The protein is photoderivatized by adding the BBA-EAC-NOS crosslinking agent at a ratio of 10-15 moles of BBA-EAC-NOS per mole of protein.

Bioactive molecules also include leptin, leukemia inhibitory factor (LIF), RGD peptide, tumor necrosis factor alpha and beta, endostatin, angiostatin, thrombospondin, osteogenic protein-1, bone morphogenic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

The term “growth factor” as used herein means a bioactive molecule that promotes the proliferation of a cell or tissue. Growth factors include, but are not limited to, transforming growth factor-alpha. (TGF-α), transforming growth factor-beta. (TGF-B), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0 s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, By8, VEGF-E, granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof. Some growth factors may also promote differentiation of a cell or tissue. TGF, for example, may promote growth and/or differentiation of a cell or tissue. Some preferred growth factors include VEGF, NGFs, PDGF-AA, PDGF-BB, PDGF-AB, FGFb, FGFa, and BGF.

The term “differentiation factor” as used herein means a bioactive molecule that promotes the differentiation of cells. The term includes, but is not limited to, neurotrophin, colony stimulating factor (CSF), or transforming growth factor. CSF includes granulocyte-CSF, macrophage-CSF, granulocyte-macrophage-CSF, erythropoietin, and IL-3. Some differentiation factors may also promote the growth of a cell or tissue. TGF and IL-3, for example, may promote differentiation and/or growth of cells.

In some aspects, if other optional components are added to the coating composition, it is generally desirable that the non-biodegradable polymer is the primary component in the composition. If the coating includes some biodegradable components, these components may degrade over a period of time, yet leaving the non-biodegradable polymer as the primary component of the coating.

The reagents of the coating composition, such as the polymeric materials, can be prepared in a suitable liquid, such as an aqueous or alcohol-based liquid. For example, the polymeric materials can be dissolved at concentrations in the range of about 0.1 mg/mL to about 50 mg/mL. However, more typically used concentrations are in the range of about 1 mg/mL to about 10 mg/mL.

The coating can be formed by any suitable method including dip coating, in-solution coating, and spray coating.

In the case wherein the coating included photoreactive groups, generally, the step of irradiating can be performed by subjecting the photoreactive groups to actinic radiation in an amount that promotes activation of the photoreactive group and bonding to the nanofibers of a nanofibrillar structure.

Actinic radiation can be provided by any suitable light source that promotes activation of the photoreactive groups. Preferred light sources (such as those available from Dymax Corp.) provide UV irradiation in the range of 190 nm to 360 nm. A suitable dose of radiation is in the range from about 0.1 mW/cm² to about 20 mW/cm² as measured using a radiometer fitted with a 335 nm band pass filter with a bandwidth of approximately 10 nm.

In some aspects, it may be desirable to use filters in connection with the step of activating the photoreactive groups. The use of filters can be beneficial from the standpoint that they can selectively minimize the amount of radiation of a particular wavelength or wavelengths that are provided to the coating during the activation process. This can be beneficial if one or more components of the coating are sensitive to radiation of a particular wavelength(s), and that may degrade or decompose upon exposure.

Typically, filters are identified by the wavelength of light that is permitted to pass through the filter. Two illustrative types of filters that can be used are cut-off filters and band pass filters. Generally, cut-off filters are categorized by a cut-off transmittance, at which the light transmittance is approximately 25% of the maximum transmittance. For band pass filters, a range of wavelength is identified for the filter, and the center wavelength is the midpoint of the wavelengths allowed through the filter.

Following the preparation of the coated nanofibrillar structure, a washing step can be performed to remove any excess materials that may not be covalently bonded to the surface of the nanofibers. The coated nanofibrillar structure can also be treated to sterilize the nanofibrillar structure, by, for example, further UV irradiation.

For in vitro applications and in the presence of the nanofibrillar substrate, cells are typically cultured in a liquid media that is suitable for maintaining cells or promoting the formation of a desired cell type or tissue. Various base liquid medias may be used, such as RPMI, which can be supplemented with serum, amino acids, trace elements, hormones, antibiotics, salts, buffers, growth factors (such as those described herein), and/or differentiation factors (such as those described herein).

Factors that can affect aspects of cellular function, including growth and differentiation can also be added to the liquid media. These factors can include neurotrophins, cytokines (such as interleukins), insulin-like growth factors, transforming growth factors, epidermal growth factors, fibroblast growth factors, heparin-binding growth factors, tyrosine kinase receptor ligands, platelet derived and vascular endothelial growth factors, and semaphorins.

In some cases, prior to disposing on the coated nanofibrillar surface, the cells may be kept on a feeder layer of cells. Following culturing for a period of time on the feeder layer, the cells may be transferred to a nanofibrillar cell culture article having the coatings.

The cells can be kept in contact with the nanofibrillar article for a period of time sufficient for upregulation of the VEGF receptor. In some aspect of the invention, in an in vitro method the cells are kept in contact with the nanofibrillar article for a period of time of about 4 hours or greater, about 6 hours or greater, about 8 hours or greater, about 10 hours or greater, about 12 hours or greater, about 14 hours or greater, about 16 hours or greater, or about 18 hours or greater. In one exemplary mode of practice the cells are keps on the nanofibrillar article for about 20 hours. The cells can be maintained on the nanofibrillar article for considerable periods of time, such as up to about 30 days.

Based on the experimental results provided herein, the invention also provides methods for modulating the proliferation of an endothelial cell. It was observed that cells cultured on either coated or uncoated nanofibers displayed longer doubling times as compared to those grown on non-nanofiber surfaces (see FIG. 5). Despite the longer doubling time, the endothelial cells demonstrated selective upregulation of Flk-1. Given this, the nanofiber article can be used in an in vitro or in vivo method to modulate the proliferation of endothelial cells.

Following contact with the article comprising the nanofiber structure, the contacted cells show a selective and significant increase in the expression of the VEGF receptor. As shown in the data from the experimental studies provided herein, expression the VEGF receptor Flk-1 in HUVEC was significantly and selectively upregulated as compared to the expression of VE-cadherin (a fibrin-specific receptor on endothelial cells; Bach, T. L. et al. J Biol Chem, Vol. 273, Issue 46, 30719-30728, Nov. 13, 1998), Tie-2 (an endothelial cell surface tyrosine receptor kinase; Partanen, J. et al. Mol Cell Biol. 1992 April; 12(4): 1698-1707), PECAM (CD31) (a multifunctional signal regulatory protein involved in endothelial cell adhesion and signalling; Simmons, D. L. (1990) J. Exp. Med. 171, 2147-2152), and von Willebrand Factor receptor (vWR; a membrane receptor that mediates adhesion to von Willebrand factor normally present in the vascular subendothelium, also known as GPIbx, a glycoprotein component of the GPIb-IX-V complex).

Expression of surface receptors can be determined using a variety of known techniques. Nucleic acids, proteins, antibodies directed against a cell surface receptor (such as Flk-1), and biological system having any of these compounds can be labeled with a detectable reagent or a compound having specificity for the surface receptor can be labeled with a detectable reagent and used to determine expression. Detectable moieties include compounds and compositions that can be detected by spectroscopic, biochemical, photochemical, bioelectronic, immunochemical, electrical, optical, or chemical techniques. Examples of detectable moieties include, but are not limited to, radioisotopes, e.g., ³²P ³³P, ³⁵S, chemiluminescent compounds, labeled binding proteins, heavy metal atoms, spectroscopic markers, such as fluorescent markers and dyes, linked enzymes, mass spectrometry tags, and magnetic labels.

The level of expression of mRNA corresponding to a cell surface receptor (such as Flk-1) can be detected utilizing commonly used molecular biology methods, for example, northern blotting, RT-PCR, real time quantitative PCR, high density arrays, and other hybridization methods. One preferred method for detecting the level of mRNA transcribed from cell surface receptor (such as Flk-1) is by RT-PCR. Details of RT-PCR techniques are well known and also described herein.

Another preferred method for detecting the level of mRNA transcripts obtained from more than one of the disclosed genes involves hybridization of labeled mRNA to an ordered array of oligonucleotides. Such a method allows the level of transcription of a plurality of these genes to be determined simultaneously to generate gene expression profiles or patterns.

Expression of a cell surface receptor (such as Flk-1) can also be carried out using components that specifically bind to the receptor on the cell surface, such as monoclonal antibodies. Immunoassay methods which utilize the antibodies include, but are not limited to, dot blotting, western blotting, competitive and non-competitive protein binding assays, enzyme-linked immunosorbant assays (ELISA), immunohistochemistry, fluorescence-activated cell sorting (FACS) and others commonly used.

Following a period of contact with the nanofibers, selective upregulation of a VEGF receptor can be measured. “Selective upregulation” means that increase in expression of a VEGF receptor in a cell contacted by the nanofiber substrate is significantly greater than the increase in expression of a non-VEGF receptor on the cell surface.

To illustrate selectivity for a VEGF receptor, upregulation can be observed as compared to other surface receptors. In one exemplary mode of practice, the expression of Flk-1 is compared to that of one or more of the surface receptors VE-cadherin, Tie-2, PECAM (CD31), or vWR, as noted above.

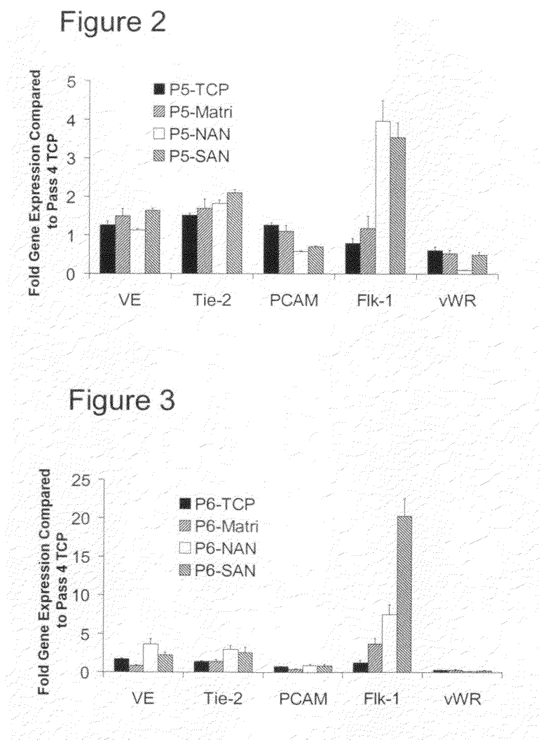

The increase in expression of Flk-1 relative to the one or more other surface receptors can be determined by measuring the relative increase in expression of the genes over a period of culturing on the nanofiber cell culture substrate. For example, gene expression can be measured over a course of cell passages on the nanofiber cell culture substrate. The expression of the genes can be determined prior to the increase in Flk-1 expression, and then at one or more points after the increase in Flk-1 expression. The fold increase in expression of Flk-1, VE-cadherin, Tie-2, PECAM (CD31), and vWR from the fourth cell passage to the fifth cell passage is illustrated in FIG. 2, and from the fourth cell passage to the sixth cell passage is illustrated in FIG. 3.

For example, the fold increase in expression of Flk-1 over one or more of VE-cadherin, Tie-2, PECAM (CD31), and vWR can be about 0.5 fold or greater (50% increase), about 1 fold or greater (100% increase), about 1.5 fold or greater (150% increase), about 2 fold or greater (200% increase), about 2.5 fold or greater (250% increase), about 3 fold or greater (300% increase), about 4 fold or greater (400% increase), about 5 fold or greater (500% increase), about 7.5 fold or greater (500% increase), or about 10 fold or greater (1000% increase).

The specificity for a nanofiber substrate, as compared to non-nanofiber substrates (such as two-dimensional tissue culture plastic and Matrigel™-coated surfaces) in the upregulation of Flk-1 is also demonstrated in FIGS. 2 and 3. For example, the fold increase in expression of Flk-1 using a nanofiber substrate over a non-nanofiber substrate can be about 1 fold or greater (100% increase), about 2 fold or greater (200% increase), about 5 fold or greater (500% increase), or about 10 fold or greater (1000% increase).

Cells cultured on nanofiber substrates and having increased expression of a VEGF receptor, such as Flk-1, can be implanted in the body in a method of cellular therapy. For example, the implanted cells can be used for tissue regeneration, such as in response to ischemia, burns, incisions, abrasions, and ulceration.

In particular, endothelial cells having upregulated Flk-1 can be implanted at a target site to promote the formation of new blood vessels. The upregulation of Flk-1 increases the is thought to promote formation and sprouting of blood vessels, termed vasculogenesis and angiogenesis, respectively.

The formation of new blood vessels, commonly seen as microvessels, is a component of the tissue healing process. An angiogenic response refers to the formation of new blood vessels from pre-existing vessels. A vasculogenic response refers to the de novo formation of new blood vessels from single cells. Exemplary sites for implantation of cultured cells include cardiac, spleen, kidney, and cerebral tissue.

The cultured cells can also be used for tissue engineering applications. Examples include the formation of artificial veins, arteries, and skin. Culturing can be carried out so the cells into a nanofiber structure capable of supporting three-dimensional tissue formation (scaffolds).

In another aspect of the invention, cells or tissue having upregulated Flk-1 can be used to screen for molecules that modulate Flk-1 receptor activity or signal transduction. Such molecules may include small organic or inorganic compounds, or other molecules that modulate Flk-1 receptor activity or that promote or prevent the formation of Flk-1/VEGF complex. Synthetic compounds, natural products, and other sources of potentially biologically active materials can be screened in a number of ways.

The ability of a test molecule to interfere with VEGF-Flk-1 binding and/or Flk-1 receptor signal may be measured using standard biochemical techniques. Other responses such as activation or suppression of catalytic activity, phosphorylation or dephosphorylation of other proteins, activation or modulation of second messenger production, changes in cellular ion levels, association, dissociation or translocation of signalling molecules, or transcription or translation of specific genes may also be monitored. These assays may be performed using conventional techniques developed for these purposes in the course of screening.

Ligand binding to its cellular receptor may, via signal transduction pathways, affect a variety of cellular processes. Cellular processes under the control of the Flk-1/VEGF signalling pathway may include, but are not limited to, normal cellular functions, proliferation, differentiation, maintenance of cell shape, and adhesion, in addition to abnormal or potentially deleterious processes such as unregulated cell proliferation, loss of contact inhibition, blocking of differentiation or cell death. The qualitative or quantitative observation and measurement of any of the described cellular processes by techniques known in the art may be advantageously used as a means of scoring for signal transduction in the course of screening.

Various embodiments are described below for screening, identification and evaluation of compounds that interact with the Flk-1 receptor, which compounds may affect various cellular processes under the control of the FLk/VEGF receptor signalling pathway.

The present invention includes a method for identifying a compound which is capable of modulating signal transduction, comprising: (a) contacting the compound with a cell having Flk-1 upregulated by the nanofiber substrate; (b) incubating the mixture of step (a) in the presence of VEGF, for an interval sufficient for the compound to stimulate or inhibit the signal transduction; (c) measuring the signal transduction in the cell; (d) comparing the signal transduction activity to that of Flk-1, incubated without the compound, thereby determining whether the compound stimulates or inhibits signal transduction.

Flk-1 signal transduction activity may be measured by standard biochemical techniques or by monitoring the cellular processes controlled by the signal. To assess modulation of kinase activity, the test molecule is added to a reaction mixture containing Flk-1 and a substrate test. To assess modulation of kinase activity of the Flk-1 receptor, the test molecule is added to a reaction mixture containing the Flk-1 receptor. The kinase reaction is then initiated with the addition of VEGF and ATP. An immunoassay is performed on the kinase reaction to detect the presence or absence of the phosphorylated tyrosine residues on the substrate or to detect phosphorylated tyrosine residues on autophosphorylated Flk-1, and results are compared to those obtained for controls i.e., reaction mixtures not exposed to the test molecule. The immunoassay used to detect the phosphorylated substrate in the cell lysate or the in vitro reaction mixture may be carried out with an anti-phosphotyrosine antibody.

The invention further provides for a method of screening compounds that, upon interacting with Flk-1, elicit or trigger a signal mimicking the action of VEGF binding to the Flk-1 receptor. Signal transduction is mimicked if the cellular processes under the control of the signalling pathway are affected in a way similar to that caused by ligand binding. Such compounds may be naturally occurring or synthetically produced molecules that activate the Flk-1 receptor.

The term “compound capable of binding to Flk-1” refers to a naturally occurring or synthetically produced molecule which interacts with Flk-1. Such a compound may directly or indirectly modulate Flk-1 signal transduction and may include molecules that are natively associated with the intracellular domain of Flk-1 inside a cell. Examples of such compounds are (i) a natural substrate of Flk-1; (ii) a naturally occurring molecule which is part of the signalling complex; and/or a naturally occurring signalling molecule produced by other cell types.

The invention also relates to the use of articles comprising a nanofiber structure in vivo. In this aspect, the expression of a VEGF receptor is selectively upregulated in a cell in vivo. In the method, an article comprising a nanofiber structure is placed at a target location in the body, wherein cells come into contact with the nanofiber which promotes the selective upregulation of the a VEGF receptor, such as Flk-1.

Desirably, the articles comprising a nanofiber is very small (particulate size or less) or degradable, and preferably very small and degradable. Exemplary articles for implantation are biodegradable nanofiber microparticles (i.e., particles having an average size of less than about 1 mm). The biodegradable nanofiber microparticles can have irregular or regular shapes. Particles can be formed by the processing of a larger nanofiber article, such as the crushing or pulverizing of a nanofiber mat. Biodegradable polymeric materials, including those described herein, can be used to the nanofiber particles.

The biodegradable nanofiber microparticles can be delivered to a site in the body wherein cell activation is desired. Exemplary sites of application include wound sites, sites at which tissue has been damaged by ischemia, and the like. Endothelial cells at the target site can contact the biodegradable nanofiber microparticles, which leads to the upregulation of Flk-1, which is thought to promote angiogenesis at the target site and restore or improve tissue function.

### EXAMPLE 1

### Culturing of Endothelial Cells on Nanofibers and Quantitative-RT-PCR of Surface Receptor Gene Expression

Nanoculture cell culture substrates were prepared as described in U.S. Publication No. 2007/0082393 (Lodhi et al.) and are commercially available from Corning (coated: Ultra-Web™ Synthetic Polyamine Surface; uncoated: Ultra-Web™ Synthetic Surface); Matrigel is commercially

Rat aortic endothelial cells (RAOECs) obtained from Cell Applications (San Diego, Calif.) were cultured in endothelial growth media provided by the manufacturer. Human umbilical vein endothelial cells (HUVECs) were obtained from BD biosciences and were cultured according to the manufacture. Cells were incubated at 37° C. in 5% CO₂/95% air humidified chamber. The media was changed every third day. Cells were trypsinized, counted and passaged when they reached 90% confluency. RAOECs and HUVECs between passage #2 and passage #8 were used for all experiments performed. The cells were trypsinized and seeded at a density of 5,000 cell/cm².

For seeding cells onto nanofibers, the RAOECs were trypsinized and resuspended in 5 ml μL complete endothelial media. The cell suspension at a density of 5,000 cell/cm² was carefully added to the Ultra-Web™ coated and uncoated nanofibers (100 mm dishes) and the cells were allowed to adhere to the nanofibers overnight. Cells were incubated at 37° C. in 5% CO₂/95% air humidified chamber. The media was changed every third day Cells were trypsinized and passaged when they reached 90% confluency and reseeded on the Ultra-Web™ 100 mm dishes.

RNA and Quantitative PCR (Q-RT-PCR). RNA was isolated using an RNeasy kit (Qiagen) with on-column DNase treatment according to the manufacture's instructions. cDNA was transcribed with random primers using Invitrogen Superscript (Invitrogen) with 0.5 μg of total RNA per reaction. Q-RT-PCR was performed in an ABI 7700 system (Applied Biosystems) using SYBR green (Applied Biosystems). Primer sets were designed for each target (Table 2) with optimal product lengths of less than 150 bp. Quantification was based on the relative expression suggested by the manufacture with GAPDH (Applied Biosystems) normalization of target genes versus passage 4 RAOECs cultured on standard tissue culture treated plastic (TCPS).

TABLE 2

| | | |
|---|---|---|
| Endothelial-Rat | Primer #1 | Primer #2 |

| | | |
|---|---|---|
| vWF | CCCACCGGATGGCTAGGTATT | GAGGCGGATCTGTTTGAGGTT |
| VE | GGCCAACGAATTGGATTCTA | GTTTACTGGCACCACGTCCT |
| GAPDH | TGCACCACCAACTGCTTAG | GATGCAGGGATGATGTTC |
| Flk1 | CCAAGCTCAGCACACAAAAA | CCAACCACTCTGGGAACTGT |
| PCAM | GGACTGGCCCTGTCACGTT | TTGTTCATGGTGCCAAAACACT |
| Tie2 | AACCAACAGTGATGTCTGGTCCTAT | GCACGTCATGCCGCAGTA |

What is claimed is:

1. A method for selectively up-regulating the expression of a VEGF receptor in a cell comprising a step of:
contacting a cell comprising a VEGF receptor gene with a synthetic nanofibrillar article for a period of time wherein expression of the VEGF receptor gene becomes selectively upregulated in the cell.

2. The method of claim 1 where the VEGF receptor gene is Flk-1.

3. The method of claim 1 where the cell is an endothelial cell.

4. The method of claim 1 wherein the synthetic nanofibrillar article comprises nanofibers comprising a polyamide condensation polymer.

5. The method of claim 1 wherein the synthetic nanofibrillar article comprises nanofibers comprising a polymeric coating on a surface of the nanofibers.

6. The method of claim 5 wherein the polymeric coating on the surface of the nanofibers comprises a non-biodegradable polymer comprising a plurality of pendent amine groups.

7. The method of claim 5 wherein the polymeric coating on a surface of the nanofibers comprises a polymer covalently bonded to the surface via reacted photo-groups.

8. The method of claim 1 wherein the step of contacting is performed in vitro.

9. The method of claim 1 wherein the step of contacting is performed for a period of time of 4 hours or greater.

10. The method of claim 9 wherein the step of contacting is performed for a period of time in the range of 4 hours to 30 days.

11. The method of claim 3 wherein the endothelial cell comprises non-VEGF receptor genes VE-cadherin, Tie-2, von Willebrand Factor receptor (vWR); and PECAM, and after the period of time of contacting the endothelial cell with the synthetic nanofibrillar article an increase in expression of the Flk-1 gene is greater than any increase in expression of the VE-cadherin, Tie-2, von Willebrand Factor receptor (vWR), or PECAM genes.

12. The method of claim 11 wherein the increase in expression of the Flk-1 gene is 0.5 fold or greater than any increase in expression of the VE-cadherin, Tie-2, von Willebrand Factor receptor (vWR), or PECAM genes.

13. The method of claim 3 wherein after the period of time of contacting endothelial cells with the synthetic nanofibrillar article, the endothelial cells have a phenotype which comprises reduced stress fibers with fiber alignment between multiple endothelial cells.

14. The method of claim 3 wherein after the period of time of contacting endothelial cells with the synthetic nanofibrillar article, the endothelial cells have a phenotype that comprises a multilayered arrangement without actin defined boarders.

15. A method for treating a medical condition in a subject comprising steps of:
providing cells comprising a VEGF receptor gene,
contacting the cells with an synthetic nanofibrillar article for a period of time wherein expression of the VEGF receptor gene becomes selectively upregulated in the cells, and
implanting the cells in which the VEGF receptor gene has become selectively upregulated in a subject to treat a medical condition.

16. The method of claim 15 wherein the cells are endothelial cells and the VEGF receptor gene is Flk-1.

17. The method of claim 15 wherein the cells in which the VEGF receptor gene has become selectively upregulated are implanted in a subject having experienced an ischemic event, tachycardia, atherosclerosis, hypotension, and/or thromboembolism.

18. The method of claim 15 wherein the cells in which the VEGF receptor gene has become selectively upregulated are implanted at an ischemic tissue site selected from the group consisting of cardiac tissue, cerebral tissue, kidney tissue, lung tissue, and spleen tissue.

19. The method of claim 15 wherein the cells in which the VEGF receptor gene has become selectively upregulated are implanted in a subject to promote an angiogenic or vasculogenic response.

20. A method for identifying a compound that affects or interacts with cells wherein a VEGF receptor has been selectively upregulated, comprising steps of:
providing cells comprising a VEGF receptor,
contacting the cells with a synthetic nanofibrillar article for a period of time wherein expression of the VEGF receptor gene becomes selectively upregulated in the cells,
applying a compound to the cells in which expression of the VEGF receptor gene is selectively upregulated, and
determining if the compound affects or interacts with cells in which expression of the VEGF receptor gene is selectively upregulated as compared to a control.

21. The method of claim 20 wherein the cells are endothelial cells and the VEGF receptor gene is Flk-1.

22. The method of claim 20 wherein the step of determining comprises determining if the compound binds to Flk-1.
